(19) United States
(12) Patent Application Publication
(10) Pub. No.: US 2024/0226185 A1
(43) Pub. Date: Jul. 11, 2024

(54) MIXTURE FOR TREATMENT OF RESPIRATORY ILLNESSES

(71) Applicant: Jack T. King, Burns Flat, OK (US)

(72) Inventor: Jack T. King, Burns Flat, OK (US)

(21) Appl. No.: 18/513,032

(22) Filed: Nov. 17, 2023

Related U.S. Application Data
(60) Provisional application No. 63/438,394, filed on Jan. 11, 2023.

(51) Int. Cl. A61K 35/644; A61K 9/08; A61K 31/045; A61K 31/121; A61K 36/534; A61K 47/02; A61K 47/10

(52) U.S. Cl. CPC A61K 35/644; A61K 9/08; A61K 31/045; A61K 31/121; A61K 36/534; A61K 47/02; A61K 47/10

(57) ABSTRACT

For the purpose of treating a patient having Covid-19 (or another respiratory illness), a composition is formed from a pair of solutes via batch processing for inhalation by a person suffering from a respiratory illness. A first solute is formed by dissolving menthol crystals into high content ethanol. A second solute is formed by dissolving Manuka honey into normal saline. After mixing the two solutes separately, the two solutes may be mixed together and the final mixture may be administered to a human person via a nebulizer.

### REFERENCE TO RELATED APPLICATIONS

This application claims priority to provisional patent application 63/438,394 filed Jan. 11, 2023 entitled Ethyl Alcohol Manuka Honey for Treatment of Respiratory Illnesses, and which is incorporated in its entirety in the present application.

### BACKGROUND OF THE INVENTION

This invention relates generally to inhaled pharmaceutical treatments for respiratory illnesses and, more particularly, a solution containing free grain alcohol and manuka honey for inhalation, such as using a nebulizer, by a patient who has been diagnosed with Covid-19 or another respiratory illness. Further, the present invention may be inhaled prophylactically by healthcare workers who are tasked with treating patients having respiratory illnesses.

With the recent global pandemic of the COVID-19 virus, a heightened level of attention is being paid to respiratory illnesses and promising methods and pharmaceuticals for treating them. One such respiratory illness that is far too common is acute pulmonary edema, also known as fluid on the lungs. Although the patient's lung function may be the first symptom, the root cause of pulmonary edema may be congestive heart failure. When the heart is unable to pump efficiently, blood may back up into the veins that travel to the lungs. As the pressure in these blood vessels increases, fluid may be pushed into the alveoli (air spaces) in the lungs. The patient may present with symptoms such as feelings of suffocation, drowning, severe shortness of breath, and even producing foam and the nose and mouth.

Fortunately, it has been found in practice and in the literature that high doses of alcohol, when inhaled (such as via a nebulizer), is very effective at reducing the foaming and other symptoms of pulmonary edema.
Inhalation of a vapor obtained by oxygen bubbling through 50% ethyl alcohol-water solution is being used for the treatment of pulmonary edema that has resisted all other forms of therapy. In pulmonary edema there is a transudation of blood elements through altered alveolar capillary walls into the alveoli, and this fluid is churned into a froth by the respiratory activity. The proponents of alcohol vapor inhalation in the treatment of pulmonary edema believe that there is an alteration of the surface tension in the alveoli producing a collapse of the foam bubbles and that the alcohol vapor penetrates into the finer air spaces. (Treatment of Pulmonary Edema, JAMA, 1954).

In addition, other respiratory illnesses may be the result of an infection causing severe inflammation. One treatment of inflammation is with manuka honey. Foraged by bees in New Zealand, manuka honey has been shown to reduce inflammation and is, therefore, at least a component of medical treatment of inflammation and bacteria I:
Manuka honey is long known to have wide ranging medicinal properties, but more recently has been identified for its broad-spectrum antimicrobial activity. Now scientists have found that manuka honey has the potential to kill a number of drug resistant bacterial infections such as Mycobacterium abscessus—which usually affects patients with cystic fibrosis (CF) or bronchiectasis. (Manuka Honey May Help Clear Drug-Resistant Lung Infection, Ashton University 2022).

Still another treatment for inflammation and airway discomfort is with menthol. More particularly, menthol can reduce airway pain and irritation when inhaled.

Therefore, it would be desirable to treat the Covid-19 infection using a combination of ethanol grain alcohol, manuka honey, and menthol so as to take advantage of the medicinal aspects of each.

### DESCRIPTION OF THE PREFERRED EMBODIMENT

An ethyl grain alcohol, manuka honey, and menthol mixture for the treatment of a patient with a respiratory illness according to a preferred embodiment of the present invention will now be described in detail. Specifically, this invention is directed to a nebulizer medicine for treating respiratory infections such as COVID-19, its variations, RSV, pulmonary edema, and the like.

Ethyl grain alcohol is the primary and preferred ingredient of the mixture according to the present invention. Ethyl alcohol, when administered in a controlled and precise manner, has the effect on bronchodilation and it relaxes smooth muscle fibers in the airways. This medical effect can facilitate improved airflow and alleviate respiratory distress in patients with conditions characterized by pulmonary edema. Further, ethyl alcohol can decrease the surface tension of alveoli within the lungs. This property may be particularly beneficial in cases of pulmonary edema, where excess fluid in the alveoli can impair gas exchange. By lowering the surface tension, ethyl alcohol may help to mitigate hypoxia and improve oxygenation. In addition, high content ethyl alcohol possesses potent anti-microbial properties. When administered in specific formulations, it can address bacterial or fungal infections that may exacerbate respiratory conditions. Of course, the administration of high content ethyl alcohol for respiratory conditions should be meticulously controlled, with dosages carefully calibrated to avoid potential toxicity and adverse effects. This monitoring would include looking for potential signs of alcohol-related side effects.

In a specific embodiment, Everclear™ is the brand name of a high content alcoholic beverage and sometimes used as a disinfectant or cleaner. Everclear is a non-denatured mixture of ethyl alcohol (ethanol, grain alcohol) product derived from 100% corn. 190 proof Everclear is designed and sold in some states for use as a beverage. That said, this type of alcohol, when inhaled via a nebulizer, has been shown to be effective in reducing the effects of respiratory illness, such as the foaming from the mouth and nasal regions of a patient. Preferably, 95% of the composition described below will be ethyl alcohol. More particularly, 95% ethyl alcohol means 95 mL out of a 100 mL liquid composition is methyl alcohol. Further, it is understood that ethyl alcohol and ethanol are terms that are used for the same liquid combination.

Manuka honey is a selected ingredient of the present invention because of its remarkable anti-inflammatory and anti-bacterial properties. Derived from the nectar of the Manuka trees blossoms, and traditionally found in New Zealand, this honey is rich in methylglyoxal (MGL), a compound responsible for its potent antibacterial effects. Studies have shown that the Manuka honey exhibits a broad-spectrum antibacterial activity against a wide range of pathogens, including antibiotic-resistant strains like MRSA.

In addition, its anti-inflammatory properties stem from its ability to reduce the production of pro-inflammatory cytokines making it a natural remedy for inflammatory conditions. Manuka honey has demonstrated its efficacy in wound healing, soothing sore throats, and alleviating gastrointestinal issues and offers a holistic approach to health and well-being.

Stated another way, Manuka honey is a type of honey that originates from certain parts of Australia and New Zealand and is made primarily from the nectar of Manuka flowers. It is understood that the nectar is largely collected by bees and their beehives. Manuka honey is considered a monofloral honey, and includes real and active ingredient known as methylglyoxal (MGO). Much more detail is available regarding the medicinal properties of manuka:
#It has been suggested that many of the medicinal properties of plants can be transmitted through honey, so that honey could be used as a vehicle for transporting plant medicinal properties. Within monofloral honey, manuka honey, a dark honey, has greatly attracted the attention of the international scientific community for its biological properties, especially for its antimicrobial and antioxidant capacities. This honey is derived from the manuka tree, Leptospermum scoparium, of the Myrtaceae family, which grows as a shrub or a small tree throughout New Zealand and eastern Australia. In traditional medicine, different extracts of the manuka tree are used as sedatives and wound-healing remedies. Moreover, manuka honey itself has long been employed for clearing up infections, including abscesses, surgical wounds, traumatic wounds, burns and ulcers of different etiology. Currently, the main bioactive compounds in manuka honey and the mechanisms responsible for their biological activities are being studied. These studies would support the increased use of manuka honey in skin medicine, and they can also be the basis for the isolation and purification of compounds for the development of bio-pharmaceutical products with antimicrobial properties and wound healing properties; these new findings could represent an added economic value that can favor also the beekeepers in their productions. (See The Composition and Biological Activity of Honey: A Focus on Manuka Honey, Food, 2014).

In another critical aspect according to the preferred embodiment of the present invention, the inventive mixture may include a predetermined quantity of menthol. Menthol is a naturally occurring compound found in various plants and, more particularly, in various mint species. As will be discussed below, the addition of menthol enhances the anti-inflammatory and antimicrobial qualities that are advantageous when treating respiratory illnesses such as pulmonary edema.

Menthol is a preferred component of the present invention because of its anti-inflammatory, antifungal and antiviral properties. Menthol acts by inhibiting the activity of certain inflammatory enzymes, such as cyclooxygenase-2 (COX-2) and LIFO oxygenase, both of which are key mediators of the inflammatory response. Further, menthol provides a cooling sensation that can help alleviate pain associated with inflammation. It achieves this by modulating the cold receptors on the skin, providing a temporary numbing effect. Moreover, menthol has relaxing properties that can ease muscle tension and spasms associated with inflammation.

For the reasons stated above, Menthol may be used in the treatment of respiratory illnesses. More particularly, menthol has a bronchodilator effect, namely, it can help open a person's airways which may become narrow and inflamed. This property is particularly valuable in treating pulmonary disease where airflow obstruction is a significant problem. Further, menthol can help loosen it as phlegm in the respiratory tract. This makes menthol even more beneficial for individuals suffering from bronchitis, pneumonia, or other respiratory illnesses. Research has shown that menthol in an amount up to 16% of the weight of a mixture is FDA approved. However, a preferred amount of menthol for the present application is about 1% to 2%.

Menthol is an organic compound, otherwise known as a monoterpenoid that is made synthetically or otherwise obtained from the oils of corn mint, peppermint, or other mint plant. Further, menthol is a waxy, clear, or white crystalline substance that dissolves at a temperature just above room temperature.

In an embodiment, a batch formula having two solutes may be mixed together to form the inventive composition. Batch processing is a manufacturing method for producing items requiring multiple steps for completion. Preferably, the elements are combined in a pair of solutes before a final combination is made. In general, it is understood that menthol crystals may be concentrated in a range from 1% to 10% of the total weight of the composition. Further, the Manuka honey is present at a concentration ranging from 1% to 15% of the total weight of the composition. Regarding the solution process, when one substance dissolves into another, a solution is formed. A solution is a homogenous mixture consisting of a solute dissolved into a solvent. The solute is the substance that is being dissolved, while the solvent is the dissolving medium. Solutions can be formed with many different types and forms of solutes and solvents.

In a preferred embodiment, the batch includes 100 mL of 95% ethanol, 1 gram of menthol crystals, 20 mL of Manuka honey, and 50 mL of normal saline. This combination provides about 1% menthol in the overall mixture. Preferably, however, a first solute is formed by dissolving the menthol crystals into the ethanol in the amounts stated previously. Further, the second solute is formed by dissolving the Manuka honey into the normal saline. After mixing the two solutes separately, the two solutes may be mixed together. The final mixture may be administered to a human person via a nebulizer. A nebulizer is a machine that turns a liquid medicine, into a fine mist which is then directed toward the airways of a patient, such as via a mask or a mouthpiece. A patient may breathe deeply through the nose or mouth or both so that the fine mist is inhaled into the bronchial tubes and beyond. In the present instance, a patient who is suffering from Covid-19, RSV, pulmonary edema, or other respiratory infections may be treated by breathing in the fine mist generated from the combination of ethyl grain alcohol with manuka honey, and menthol.

It is understood that while certain forms of this invention have been illustrated and described, it is not limited thereto except insofar as such limitations are included in the following claims and allowable functional equivalents thereof.

1. A method for treating a respiratory condition, comprising:
(a) dissolving methanol crystals into ethyl alcohol to form a first solute;
(b) dissolving manuka honey into saline solution to form a second solute; and
(c) mixing the first solute in the second solute together to form a composition.

2. The method as in claim 1, further comprising administering an effective amount of the composition to a patient in need thereof using a nebulizer.

3. The method as in claim 1, wherein the menthol crystals are present in a concentration ranging from 1% to 10% of the total weight of the composition.

4. The method as in claim 1, wherein the manuka honey is present in a concentration ranging from 1% to 15% of the total weight of the composition.

5. The method as in claim 1, wherein the ethyl alcohol is 95% ethyl grain alcohol.

6. The method as in claim 5, wherein the ethyl alcohol includes 50% to 95% ethanol.

7. The method as in claim 1, wherein:
said ethyl alcohol includes 100 mL of 95% ethanol;
said menthol crystals includes 1 g of menthol crystals;
said the Manuka honey includes 20 mL of Manuka honey; and
said saline solution includes 50 mL of saline solution.

8. The method as in claim 1, wherein said Manuka honey includes a concentration of methylglyoxal (MGO) of at least 100 mg/kg.

9. The method as in claim 1, wherein said menthol crystals are derived from natural plant sources.

10. The method as in claim 1, wherein said menthol crystals are derived from peppermint oil.

11. A composition for treating a patient having a respiratory illness, comprising:
a first solute formed by dissolving menthol into ethyl alcohol;
a second solute or by dissolving Manuka honey into saline solution;
wherein the first solute and second solute are mixed together.

12. The composition as in claim 11, further comprising a nebulizer configured to receive said composition and operable to administer that composition to the patient.

13. The composition as in claim 12, wherein the menthol crystals are present in a concentration ranging from 1% to 10% of the total weight of the composition.

14. The composition as in claim 12, wherein the Manuka honey is present in a concentration ranging from 1% to 15% of the total weight of the composition.

15. The composition as in claim 12, wherein the ethyl alcohol is 95% ethyl grain alcohol.

16. The composition as in claim 11, wherein:
said ethyl alcohol includes 100 mL of 95% ethanol;
said menthol crystals includes 1 g of menthol crystals;
said the manuka honey includes 20 mL of manuka honey; and
said saline solution includes 50 mL of saline solution.

17. The composition as in claim 11, wherein said Manuka honey includes a concentration of methylglyoxal (MGO) of at least 100 mg/kg.

18. The composition as in claim 11, wherein said menthol crystals are derived from natural plant sources.

19. The composition as in claim 18, wherein said menthol crystals are derived from peppermint oil.
